(19) United States
(12) Patent Application Publication
(10) Pub. No.: US 2010/0305414 A1
(43) Pub. Date: Dec. 2, 2010

(54) METHOD AND APPARATUS FOR TRANSMITTING BIOLOGICAL INFORMATION OF USER

(75) Inventors: Yoon-seo KOO, Yongin-si (KR); Hong-sig KIM, Yongin-si (KR); Jun-il SOHN, Yongin-si (KR)

Correspondence Address: CANTOR COLBURN LLP, 20 Church Street, 22nd Floor, Hartford, CT, 06103, US

(73) Assignee: SAMSUNG ELECTRONICS CO., LTD., Suwon-si (KR)

(21) Appl. No.: 12/723,932

(22) Filed: Mar. 15, 2010

(30) Foreign Application Priority Data
May 26, 2009 (KR) 10-2009-0046147

(51) Int. Cl. A61B 5/00; H04B 1/04

(52) U.S. Cl. 600/301; 455/127.5

(57) ABSTRACT

A method of transmitting biological information includes selecting a transmission mode from a plurality of transmission modes and transmitting biological information of a user to an external device using the selected transmission mode. Each of the transmission modes transmits different quantities of data to the external device based on a state of the biological information of the user.

### CROSS-REFERENCE TO RELATED APPLICATION

This application claims priority to Korean Patent Application No. 10-2009-0046147, filed on May 26, 2009, and all the benefits accruing therefrom under 35 U.S.C. §119, the content of which in its entirety is herein incorporated by reference.

### BACKGROUND

1) Field

The general inventive concept relates to a method and an apparatus for transmitting biological information. More particularly, the general inventive concept relates to a method and an apparatus for transmitting biological information of a user, in which power consumption is significantly reduced.

2) Description of the Related Art

In addition to patient monitoring devices used in hospitals, a variety of wearable medical devices, such as portable electrocardiography (“ECG”) measuring devices and portable pulse oximeters, for example, which measure biological signals of users outside a hospital, have been developed. These wearable medical devices transmit biological information of the users to peripheral devices, such as cellular phones, personal computers (“PCs”) and tele-monitoring devices, typically in a wireless manner. By removing wired connections between the wearable medical devices and the peripheral devices, performance of the wearable medical devices and convenience of the users are improved, and a size of the devices can be reduced. However, as the wearable medical devices become smaller, the size and weight of batteries used in the wearable medical devices become more restricted.

### SUMMARY

Provided is a method and apparatus for substantially reducing power consumption of a wearable medical device that measures and transmits biological information of a user, to thereby reduce a required size of the wearable medical device and/or of a battery used therewith. Also provided is a computer readable recording medium recording a program for executing the method.

Provided is a method of transmitting biological information that includes selecting a transmission mode from a plurality of transmission modes and transmitting biological information of a user to an external device using the selected transmission mode. Each of the transmission modes transmits different quantities of data to the external device based on a state of the biological information of the user.

Provided also is a computer program product including a computer readable computer program code for executing a method of transmitting biological information, and instructions for causing a computer to implement the method. The method includes selecting a transmission mode from a plurality of transmission modes and transmitting biological information of a user to an external device using the selected transmission mode. Each of the transmission modes transmits different quantities of data to the external device based on a state of the biological information of the user.

Also provided is an apparatus for transmitting biological information, which includes: a controller which selects a transmission mode from a plurality of transmission modes, each of the transmission modes transmitting different quantities of data to an external device based on a state of biological information of a user; and a transmitter which transmits the biological information of the user to the external device using the selected transmission mode.

### BRIEF DESCRIPTION OF THE DRAWINGS

The above and/or other aspects will become more readily apparent and more readily appreciated from the following description of example embodiments, taken in conjunction with the accompanying drawings, in which:

FIG. 1 is a block diagram of an example embodiment of a healthcare system for a user;

FIG. 2 is a block diagram of an example embodiment of a biological information transmission device of the healthcare system shown in FIG. 1;

FIGS. 3A, 3B, 4A and 4B are graphs of signal intensity versus time, which illustrate transmission modes selected by a controller of the healthcare system shown in FIG. 1; and

FIG. 5 is a flowchart illustrating an example embodiment of a method of transmitting biological information of a user.

### DETAILED DESCRIPTION

The general inventive concept now will be described more fully hereinafter with reference to the accompanying drawings, in which various example embodiments are shown. The general inventive concept may, however, be embodied in many different forms, and should not be construed as limited to the example embodiments set forth herein. Rather, these example embodiments are provided so that this disclosure will be thorough and complete, and will fully convey the scope of the general inventive concept to those skilled in the art Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the general inventive concept.

The terminology used herein is for the purpose of describing particular example embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated regions, integers, steps, operations, elements, and/or components, but do not preclude the presence or addition of one or more other regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Example embodiments are described herein with reference to cross section illustrations that are schematic illustrations of idealized example embodiments. As such, variations from the shapes of the illustrations as a result, for example, of manufacturing techniques and/or tolerances, are to be expected. Thus, example embodiments described herein should not be construed as limited to the particular shapes of regions as illustrated herein but are to include deviations in shapes that result, for example, from manufacturing. For example, a region illustrated or described as flat may, typically, have rough and/or nonlinear regions. Moreover, sharp angles that are illustrated may be rounded. Thus, the regions illustrated in the figures are schematic in nature and their shapes are not intended to illustrate the precise shape of a region and are not intended to limit the scope of the present claims.

Hereinafter, example embodiments of the general inventive concept will be described in further detail with reference to the accompanying drawings.

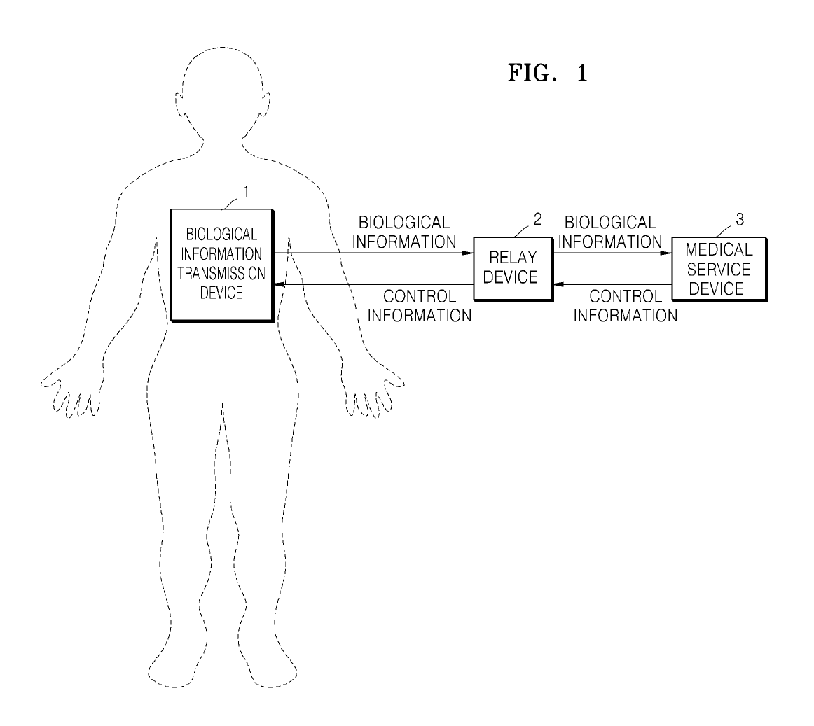

FIG. 1 is a block diagram of an example embodiment of a healthcare system of a user. Referring to FIG. 1, the healthcare system of the user according to one or more example embodiments includes a biological information transmission device 1, a relay device 2 and a medical service device 3. The biological information transmission device 1 may be a medical device wearable by the user, for example. Specific examples of the biological information transmission device 1 include a portable electrocardiography (“ECG”) measuring device and a portable pulse oximeter, but additional example embodiments are not limited thereto. The relay device 2 relays communications between the biological information transmission device 1 and the medical service device 3. Examples of the relay device 2 include a cellular phone, a personal computer (“PC”) and a tele-monitoring apparatus, for example. Communication between the biological information transmission device 1 and the relay device 2 may be performed using radio frequency (“RF”) and/or a body area network (“BAN”), for example. In addition, communication between the relay device 2 and the medical service device 3 may be performed using a local area network (“LAN”), a wireless local area network (“WLAN”) and/or code division multiple access (“CDMA”), although additional example embodiments are not limited to the foregoing communication devices or methods.

Biological information of the user is transmitted from the biological information transmission device 1 to the relay device 2, and is transmitted from the relay device 2 to the medical service device 3. In addition, information for controlling the transmission of the biological information of the user may be transmitted from the relay device 2 to the biological information transmission device 1, and/or transmitted from the medical service device 3 to the biological information transmission device 1 via the relay device 2. When the control information is set by the user, the control information is transmitted from the relay device 2 to the biological information transmission device 1. When the control information is set by a medical expert, such as a physician, for example, the control information is transmitted from the medical service device 3 to the biological information transmission device 1 via the relay device 2.

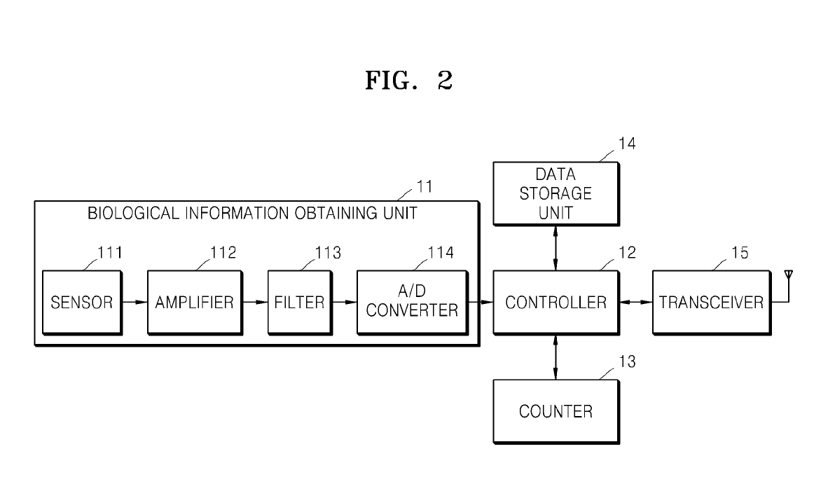

FIG. 2 is a block diagram of an example embodiment of the biological information transmission device 1 shown in the healthcare system of FIG. 1. Referring to FIG. 2, the biological information transmission device 1 includes a biological information obtaining unit 11, a controller 12, a counter 13, a data storage unit 14 and a transceiver 15. The biological information obtaining unit 11 obtains the biological information of the user. The biological information obtaining unit 11 includes a sensor 111, an amplifier 112, a filter 113 and an analog/digital (“A/D”) converter 114. The sensor 111 measures one or more biological signals of the user. The amplifier 112 amplifies the measurement of the biological signal of the user measured by the sensor 111. The filter 113 removes noise and other signals that are not biological information from the measurement of the biological signals amplified by the amplifier 112. The A/D converter 114 converts the biological signal, filtered by the filter 113, from an analog signal into a digital signal to obtain the biological information of the user.

The controller 12 resets a count value of the counter 13 to zero (0) when the biological information transmission device 1 is powered on, to initiate operation of the biological information transmission device 1, and/or at a predetermined interval. The count value is used to measure a data transmission time of the biological information transmission device 1, as will be described in further detail below.

The controller 12 selects one transmission mode, of a plurality of transmission modes, in which different quantities of data are transmitted to the relay device 2 based on a state of the biological information obtained by the biological information obtaining unit 11. More specifically, the controller 12 identifies whether an event indicating an abnormal state of the biological information obtained by the biological information obtaining unit 11 is generated. For example, the controller 12 compares values indicating the state of the biological information obtained by the biological information obtaining unit 11 to corresponding threshold values, and identifies a generation of an event based on results of the comparison. The threshold values are set by the user of the relay device 2 or the medical expert using the medical service device 3, so that the healthcare for the user is efficiently managed, and customized medical devices may thereby be easily fabricated.

More specifically, when the event is not generated for a predetermined amount of time, the controller 12 selects one of the transmission modes, e.g., a first transmission mode, in which a first, relatively small, quantity of data is transmitted to the relay device 2. In contrast, the controller 12 selects another one of the transmission modes, e.g., a second transmission mode, in which a second quantity of data (relatively large compared to the first quantity of data of the first transmission mode) is transmitted to the relay device 2 when the generation of the event is identified. In one or more example embodiments, the controller 12 may select the transmission modes according to control information received by the transceiver 15.

According to an example embodiment, the abnormal state of the biological information of the user may indicate either an abnormal state of the user's health or an abnormal state of the transmission of the biological information of the user. It will be understood, however, that numerous other abnormal states, other than the abnormal states described herein, may be indicated in additional example embodiments.

In one or more example embodiments, when the event is generated, the biological information transmission device 1 may inform the medical service device 3 of the generation of the event via the relay device 2, and all of the biological information of the user may thereafter be transmitted.

The controller 12 utilizes the count value, counted by the counter 13, to identify whether the event has been generated for the predetermined amount of time. Specifically, the counter 13 increases the count value, which is periodically reset to 0 by the controller 12, as discussed above, by 1 count per second, for example, but not being limited thereto. The data storage unit 14 stores biological information obtained by the biological information obtaining unit 11 but not transmitted to the relay device 2. In an example embodiment, the data storage unit 14 may be a memory, such as a flash memory, for example. The biological information stored in the data storage unit 14 may be downloaded to the relay device 2 in a wireless or a wired manner, such as by a universal serial bus (“USB”) interface, for example, since the biological information stored in the data storage unit 14 may be too large to be efficiently transmitted to the relay device 2 in the wireless manner. It will be noted that various additional methods of reducing the transmission rate of large sized biological information, other than described herein, may be used in additional example embodiments.

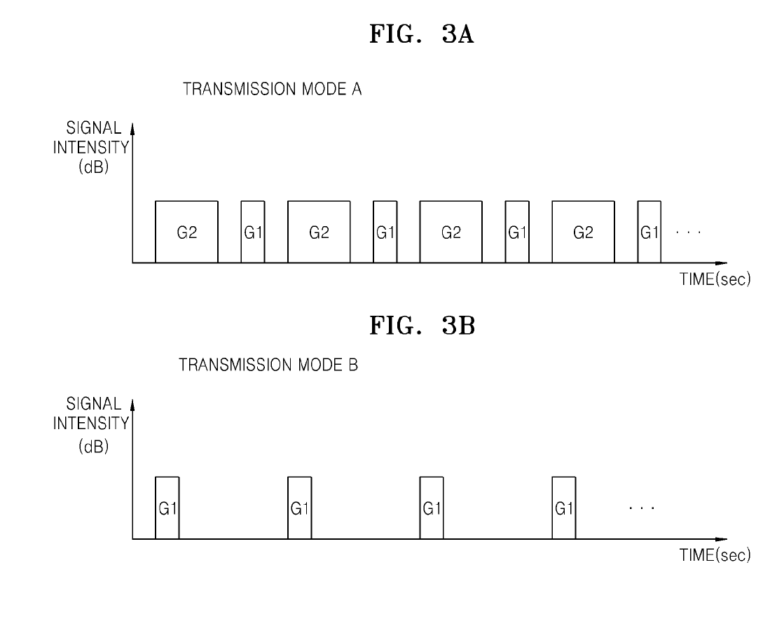

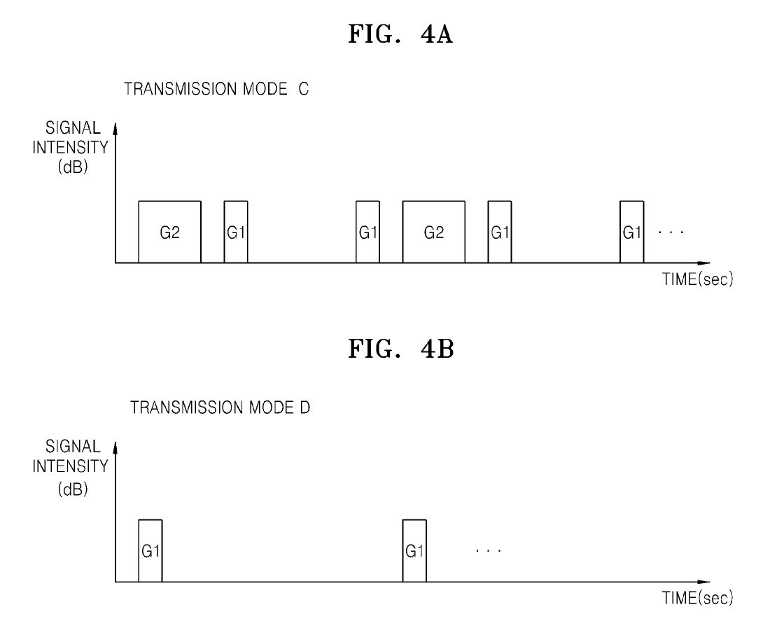

FIGS. 3A, 3B, 4A, and 4B are graphs of signal intensity, in decibels (dB), versus time, in seconds (sec), which illustrate example embodiments of transmission modes that the controller 12 shown in FIG. 1 selects. Specifically, FIGS. 3A and 3B illustrate transmission modes A and B, respectively, by which the biological information of the user is classified into a first group G1 and a second group G2. The first group G1 contains biological information that has relatively small data size (relative to the second group G2) and a relatively high importance (relative to the second group G2) and which may be used as an index of the state of the user's health. Likewise, the second group G2 contains biological information that has relatively large data size and relatively low importance (relative to the first group G1), and which is not used as an index of the state of the user's health.

FIG. 3A shows the transmission mode A, in which all of the biological information obtained by the biological information obtaining unit 11, e.g., all of the biological information in both the first group G1 and the second group G2, is transmitted to the relay device 2 while equalizing the transmission frequency of the biological information. The controller 12 provides all of the biological information obtained by the biological information obtaining unit 11 to the user (and/or the medical expert) when an abnormal health state of the user, or an abnormal transmission state of the biological information, is identified, e.g., when the generation of the event is identified. In this case, the controller 12 selects the transmission mode A.

FIG. 3B shows the transmission mode B, in which a portion of the biological information obtained by the biological information obtaining unit 11, e.g., only the biological information of the first group G1, is transmitted to the relay device 2. The controller 12 does not provide all of the biological information obtained by the biological information obtaining unit 11 to the user or the medical expert, but instead only provides a minimum amount of information to determine whether the user is in an abnormal health state, when the event is not generated for the predetermined amount of time. In this case, the controller 12 selects the transmission mode B.

FIGS. 4A and 4B illustrate transmission modes C and D, which have varied transmission frequencies for each portion of the biological information obtained by the biological information obtaining unit 11. Specifically, FIG. 4A illustrates the transmission mode C, in which the biological information obtained by the biological information obtaining unit 11 is transmitted to the relay device 2 having a varied transmission frequency of each part of the biological information, e.g., having a varied the transmission frequency of the first group G1 and a varied transmission frequency of the second group G2. As shown in FIG. 4A, the transmission frequency of the first group G1 is twice that of the second group G2.

FIG. 4B illustrates the transmission mode D, in which the first group G1 (of the transmission mode B of FIG. 3B) is transmitted to the relay device 2 having varied the transmission frequency of the first group G1. As shown in FIG. 4B, the transmission of the first group G1 is less frequently performed (relative to FIG. 3B). However, in one or more additional embodiments, the transmission of the first group G1 may be more frequently performed than shown in FIGS. 3B and 4B.

Referring still to FIGS. 3A, 3B, 4A, and 4B, and comparing the transmission modes A, B, C and D shown therein, it can be seen that transmission mode A (FIG. 3A) has the largest quantity of data transmitted to the relay device 2, while transmission mode D (FIG. 4B) has the smallest quantity of data transmitted to the relay device 2. Thus, according to one or more example embodiments, the transmission quantity of the biological information, e.g., the duty cycle of wirelessly transmitted data, may be adaptively controlled by transmitting the biological information using one of the transmission modes A, B, C and D shown in FIGS. 3A, 3B, 4A and 4B, respectively, based on a state of the biological information obtained by the biological information obtaining unit 11. Accordingly, power consumption of the biological information transmission device 1 is substantially reduced. Specifically, for example, when the duty cycle of the wirelessly transmitted data is reduced from 30 percent (%) to 10%, the power consumption of the biological information transmission device 1 is reduced by about ⅓, e.g., by about 33%. Thus, the duty cycle indicates a ratio of a wireless data transmission portion to the entire data transmission.

In one or more example embodiments, the transceiver 15 transmits the biological information, obtained by the biological information obtaining unit 11, to the relay device 2. More particularly, the transceiver 15 generates a high power RF signal to transmit the biological information obtained by the biological information obtaining unit 11 in a wireless manner, and a large amount of power is thereby required. Thus, by reducing a quantity of the data to be transmitted in the wireless manner by the transceiver 15, the power consumption of the biological information transmission device 1 is efficiently reduced. To reduce the quantity of data transmitted in the wireless manner by the transceiver 15, one of the various transmission modes A, B, C or D, shown in FIGS. 3A, 3B, 4A and 4B, respectively, is selected. The transceiver 15 transmits the biological information obtained by the biological information obtaining unit 11 to the relay device 2 using one of the transmission modes A, B, C and D selected by the controller 12.

As discussed above, when the controller 12 selects transmission mode A, the transceiver 15 transmits all of the biological information obtained by the biological information obtaining unit 11 to the relay device 2 while equalizing the transmission frequency of each part of the biological information. When the controller 12 selects transmission mode B, the transceiver 15 transmits only a portion of the biological information obtained by the biological information obtaining unit 11 to the relay device 2, according to a priority based on characteristics of the each portion of the biological information. For example, as shown in FIG. 3, the first group G1 has relatively small data size and relatively high importance and may be used as an index of the state of the user's health, and therefore the first group G1 has a higher priority than that of the second group G2. Accordingly, the transceiver 15 transmits the biological information corresponding to the first group G1 to the relay device 2.

When the controller 12 selects transmission mode C, the transceiver 15 transmits the biological information obtained by the biological information obtaining unit 11 to the relay device 2 while having varied the transmission frequency according to priority based on characteristics of each portion of the biological information. For example, since the first group G1 has a higher priority than the second group G2, the transceiver 15 transmits the biological information to the relay device 2 having an increased transmission frequency of the biological information of the first group G1, and a decreased transmission frequency of the biological information of the second group G2. When the controller 12 selects transmission mode D, the transceiver 15 transmits a portion of the biological information obtained by the biological information obtaining unit 11 to the relay device 2 while controlling the transmission frequency of the transmitted portion of the biological information based on the state of the biological information. For example, the transceiver 15 transmits the biological information of the first group G1 to the relay device 2 having an increased transmission frequency of the biological information when it is identified that the event is generated. If the event is not generated for a predetermined amount of time, the transceiver 15 transmits the biological information of the first group G1 to the relay device 2 having a reduced transmission frequency of the biological information.

In addition, the transceiver 15 receives control information for selection of transmission mode by the controller 12, from the relay device 2. The control information includes the threshold values, wireless signal intensity information measured by the relay device 2, and information for directly controlling the selection of the transmission mode by the controller 12, for example, although additional example embodiments are not limited to the foregoing list.

ECG is recording electrical variations produced by a heart, wherein the electrical variations are sensed by electrodes in contact with the skin of a user. When the biological information transmission device 1 shown in FIG. 1 is an ECG measuring device, the sensor 111 may be an electrode in contact with the user's skin. Data to be transmitted to the relay device 2 by the biological information transmission device 1 may be ECG graphic information, heart rate information, alarm information and/or wireless signal intensity information, for example. In one or more example embodiments, the ECG graphic information is transmitted at a frequency of 250 Hertz (Hz) and has a size of 10 bits. The heart rate information may be transmitted at a frequency of 1 Hz and have a size of 8 bits. The alarm information may be transmitted at a frequency of 1 Hz and have a size of 7 bits. The alarm information may include information indicating an off-state of the sensor 111, information indicating an error in sensing of the sensor 111 (such as a motion artifact, for example), and/or information indicating arrhythmia, for example. The wireless signal intensity information indicates an intensity of the wireless signal transmitted to the relay device 2 by the biological information transmission device 1.

ECG is a method used to monitor the state of the heart of a patient. For a precise diagnosis of the heart, waveforms of an ECG graph are analyzed. For example, a heart rate calculated by detecting peaks of the ECG graph is generally used as an index of the state of the user's health, instead of referring to the waveforms of the ECG graph themselves. For example, for a patient without a heart disorder, the heart rate, instead of the ECG graph, is monitored. In addition, when an exertion level of a user wearing a portable medical device is measured, the heart rate is monitored. As described above, among the data to be transmitted to the relay device 2 by the biological information transmission device 1, the ECG graph information has a relatively large data size and low importance, and therefore may not be used as an index of the state of the user's health (as compared to the other information). Thus, in an example embodiment, the ECG graph information is included in the second group G2, and other information including the heart rate is included in the first group G1. When the transceiver 15 transmits the first group G1, obtained by the to the relay device 2, using the transmission mode B, the ECG graph information, for example, which is in the second group G2, is not transmitted. As a result, a data quantity rate is thereby reduced by about 300 bytes per second.

The threshold values, which are referred to by the controller 12 for selecting the transmission mode, may include maximum heart rate, minimum heart rate, heart rate variation, minimum wireless signal intensity and/or maximum transmission mode-maintaining time, for example. Thus, if the heart rate of the user indicated by the heart rate information obtained by the biological information obtaining unit 11 is greater than the maximum heart rate threshold value, or less than the minimum heart rate threshold value, the controller 12 determines that the event of an abnormal state of the biological information obtained by the biological information obtaining unit 11 is generated and selects transmission mode A. Likewise, if a heart rate variation of the user, indicated by the heart rate information obtained by the biological information obtaining unit 11, is greater than the heart rate variation threshold value, the controller 12 determines that the event is generated and selects transmission mode A.

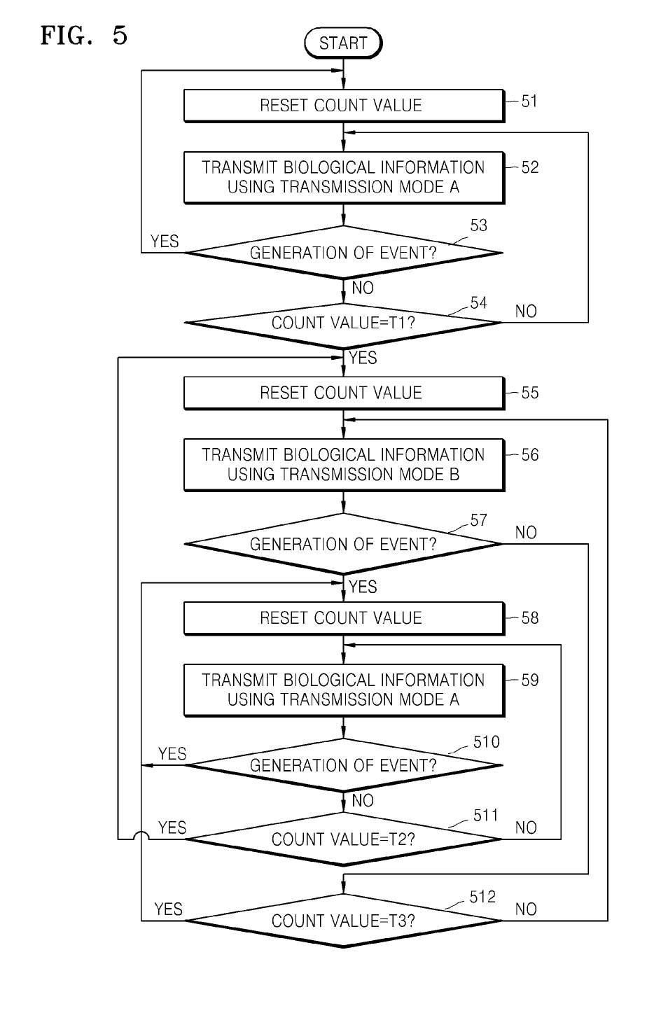

If the intensity of the wireless signal received by the relay device 2 is less than the minimum wireless signal intensity threshold value, the controller 12 determines that the event is generated and selects transmission mode A. If the wireless signal transmitted between the biological information transmission device 1 and the relay device 2 is weak, the possibility that the relay device 2 receives accurate biological information decreases, and thus, when the wireless signal is weak, all of the biological information of the user is transmitted. In addition, if the current transmission mode-maintaining time is greater than the maximum transmission mode-maintaining time threshold value, the controller 12 changes the transmission mode, as will be described in greater detail with reference to FIG. 5.

A photoplethysmograph (“PPG”) is a plethysmograph obtained using an optical method. When the biological information transmission device 1 of FIG. 1 is a pulse oximeter for performing photoplethysmography, the sensor 111 may include light emitting diodes (“LEDs”) and photodiodes in contact with a part of the body of the user, such as at the user's fingertips or earlobes, for example. Data to be transmitted to the relay device 2 by the biological information transmission device 1 may be PPG graph information, blood oxygen saturation (“SpO₂”) information, heart rate information, biological signal intensity information, alarm information and/or wireless signal intensity information, for example.

The PPG graph information may be transmitted at a frequency of 60 Hz and have a size of 8 bits. The blood oxygen saturation information may be transmitted at a frequency of 1 Hz and have a size of 7 bits. The heart rate information generally may be transmitted at a frequency of 1 Hz and have a size of 8 bits. The biological signal intensity information is classified as instant biological signal intensity information or average biological signal intensity information. The instant biological signal intensity information may be transmitted at a frequency of 60 Hz and have a size of 4 bits. The average biological signal intensity information is an average of the instant biological signal intensity information, may be transmitted at a frequency of 1 Hz, and may have a size of 4 bits. The alarm information may be transmitted at a frequency of 1 Hz and have a size of 7 bits. The alarm information may include information indicating the off-state of the sensor 111 and/or information indicating an error in sensing of the sensor 111 (such as a motion artifact), for example. The wireless signal intensity information indicates the intensity of wireless signals transmitted to the relay device 2 by the biological information transmission device 1.

A heart rate that is calculated from a PPG and blood oxygen saturation values may be used as indices indicating the state of the user's health, instead of waveforms of the PPG graph. The waveforms of the PPG are referred to in order to precisely diagnose the specific state of the user's health. However, if the accuracy of the PPG is guaranteed, the user or the medical expert does not need to monitor the PPG. As described in greater detail above, among data to be transmitted to the relay device 2 by the biological information transmission device 1, the PPG information and the instant biological signal intensity information have relatively large data quantity and relatively low importance and may not be used as an index indicating the state of the user's health. Thus, the PPG graph information and the instant biological signal intensity information are included as the second group G2, and other information, such as the heart rate, may be included as the first group G1 (as shown in FIGS. 3A, 3B, 4A and 4B). When the transceiver 15 transmits the first group G1 to the relay device 2 from among the biological information of the user obtained by using the transmission mode B, the PPG graph information and the instant biological signal intensity information are not transmitted. As a result, a data quantity rate is reduced by about 85 bytes per second.

The threshold values, referred to by the controller 12 for selecting the transmission mode, may include maximum heart rate, minimum heart rate, heart rate variation, minimum blood oxygen saturation, blood oxygen saturation variation, minimum wireless signal intensity and/or maximum transmission mode-maintaining time, for example. If the heart rate of the user indicated by the heart rate information obtained by the biological information obtaining unit 11 is greater than the maximum heart rate threshold value or less than the minimum heart rate threshold value, the controller 12 determines that the event of an abnormal state of the biological information obtained by the biological information obtaining unit 11 is generated and selects transmission mode A. If a heart rate variation of the user indicated by the heart rate information obtained by the biological information obtaining unit 11 is greater than the heart rate variation threshold value, the controller 12 determines that the event is generated and selects transmission mode A.

If the blood oxygen saturation indicated by the blood oxygen saturation information obtained by the biological information obtaining unit 11 is less than the minimum blood oxygen saturation threshold value, the controller 12 determines that the event is generated and selects transmission mode A. If a blood oxygen saturation variation of the user indicated by the blood oxygen saturation information obtained by the biological information obtaining unit 11 is greater than the blood oxygen saturation variation threshold value, the controller 12 determines that the event is generated and selects transmission mode A. If the intensity of the wireless signal received from the relay device 2 is less than the minimum wireless signal intensity threshold value, the controller 12 determines that the event is generated and selects transmission mode A. The controller 12 changes the transmission mode when the current transmission mode-maintaining time is greater than the maximum transmission mode-maintaining time threshold value, as will now be described in greater detail with reference to FIG. 5.

FIG. 5 is a flowchart illustrating an example embodiment of a method of transmitting biological information. Referring to FIG. 5, the method of managing a patient according to an example embodiment includes operations processed by the biological information transmission device 1 shown in FIG. 1. Thus, even though not described hereinafter, the biological information transmission device 1 shown in FIG. 1 is also utilized in the method of transmitting the biological information according to one or more embodiments.

In operation 51, when powering on the biological information transmission device 1 to initiate the operation of the biological information transmission device 1, for example, the biological information transmission device 1 resets the count value for measuring the transmission mode-maintaining time of the biological information transmission device 1 and selects the transmission mode A. In operation 52, the biological information transmission device 1 transmits the biological information of the user to the relay device 2 using the transmission mode A while increasing the count value by 1 count per second, for example.

In operation 53, the biological information transmission device 1 determines whether the event indicating that at least a part of the biological information transmitted by the biological information transmission device 1 is in an abnormal state is generated. As a result, if it is determined in operation 53 that the event is generated, the method proceeds back to operation 51, and if it is determined in operation 53 that the event is not generated, the method proceeds to operation 54.

In operation 54, the biological information transmission device 1 determines whether the count value has reached a first count value T1. If it is determined in operation 54 that the count value has reached the first count value T1, the method proceeds to operation 55, and if it is determined in operation 54 that the count value has not reached the value T1, the method proceeds to operation 52. In an example embodiment, the first count value T1 indicates an amount of time sufficient to transmit all of the biological information that is measured by the biological information transmission device 1.

In operation 55, the biological information transmission device 1 resets the count value to zero (0) and selects transmission mode B. In operation 56, the biological information transmission device 1 transmits the biological information of the user to the relay device 2 using the transmission mode B while increasing the count value by 1 count per second, for example.

In operation 57, the biological information transmission device 1 determines whether the event indicating that at least a part of the biological information transmitted by the biological information transmission device 1 is in an abnormal state is generated. As a result, if it is determined in operation 57 that the event is generated, the method proceeds to operation 58, and if it is determined in operation 57 that the event is not generated, the method proceeds to operation 512.

In operation 58, the biological information transmission device 1 resets the count value to zero (0) and selects transmission mode A. In operation 59, the biological information transmission device 1 transmits the biological information of the user to the relay device 2 using the transmission mode A while increasing the count value by 1 count per second, for example.

In operation 510, the biological information transmission device 1 determines whether the event indicating that at least a part of the biological information transmitted by the biological information transmission device 1 is in an abnormal state is generated. As a result, if it is determined in operation 510 that the event is generated, the method proceeds to operation 58, and if it is determined in operation 510 that the event is not generated, the method proceeds to operation 511.

In operation 511, the biological information transmission device 1 determines whether the count value has reached a second count value T2. If it is identified in operation 511 that the count value has reached the second count value T2, the method proceeds to operation 55, and if it is identified in operation 511 that the count value has not reached the second count value T2, the method proceeds to operation 59. In an example embodiment, the second count value T2 indicates an amount of time sufficient for the biological information to return to a stable state after the event of the abnormal state of the biological information of the user is generated.

In operation 512, the biological information transmission device 1 determines whether the count value has reached a third count value T3. If it is determined in operation 512 that the count value has reached the third count value T3, the method proceeds to operation 55, and if it is determined in operation 512 that the count value has not reached the third count value T3, the method proceeds to operation 59. In an example embodiment, the third count value T3 indicates an amount of time sufficient to determine that the biological information of the user is to be transmitted using the transmission mode A after the biological information of the user is transmitted using the transmission mode B.

To substantially reduce power consumption of the biological information transmission device 1 according to one or more exemplary embodiments, the transmission modes A and B, as shown in the example embodiment of FIG. 5, may be replaced by the transmission modes C and D, respectively. In addition, it will be understood that various additional transmission modes, other than the transmission modes described herein, may be used in additional example embodiments.

As described herein, a quantity of data transmitted in a wireless manner is substantially reduced by selecting one of a plurality of transmission modes, in which different quantities of data are transmitted to an external device, based on a state of biological information of a user. As a result, power consumption of a wearable medical device that transmits the biological information of the user is significantly reduced. Since the power consumption of the wearable medical device is reduced, an operation time of the device is increase, thereby substantially improving convenience to the user. In addition, a required size and weight of a battery is reduced, and thus the required size of the wearable medical device is reduced.

The example embodiments described herein may be included in or written as computer programs, and may be implemented in specific- or general-use digital computers that execute the computer programs using a computer-readable recording medium. Data used in the one or more example embodiments may be recorded by using various units in the computer-readable recording medium. Examples of the computer-readable recording medium include, but are not limited to, magnetic storage media, e.g., read only memories (“ROMs”), floppy discs, and hard discs, as well as optically readable media, such as compact disc-read only memories (“CD-ROMs”) and digital versatile discs (“DVDs”), for example.

While the general inventive concept has been particularly shown and described with reference to example embodiments thereof, it will be understood by those of ordinary skill in the art that various changes in form and details may be made therein without departing from the spirit or scope of the general inventive concept as defined by the following claims.

What is claimed is:

1. A method of transmitting biological information, the method comprising:
selecting a transmission mode from a plurality of transmission modes, each of the transmission modes transmitting different quantities of data to an external device based on a state of biological information of a user; and
transmitting the biological information of the user to the external device using the selected transmission mode.

2. The method of claim 1, wherein the selecting the transmission mode comprises selecting a transmission mode, from the plurality of transmission modes, in which a first quantity of data is transmitted to the external device, when a normal state of the biological information is maintained for a predetermined amount of time.

3. The method of claim 2, wherein the transmitting the biological information comprises transmitting a portion of the biological information to the external device according to a priority, which is based on characteristics of all portions of the biological information, using the selected transmission mode.

4. The method of claim 3, wherein the characteristics comprise at least one selected from a group consisting of data size and an importance of each portion of the biological information.

5. The method of claim 2, wherein the transmitting the biological information comprises transmitting portions of the biological information to the external device, each portion including a transmission frequency which varies according to a priority, which is based on characteristics of all portions of the biological information, using the selected transmission mode.

6. The method of claim 5, wherein the characteristics comprise at least one selected from a group consisting of data size and an importance of each portion of the biological information.

7. The method of claim 2, wherein
the selecting the transmission mode further comprises selecting a second transmission mode, from the plurality of transmission modes, in which a second quantity of data is transmitted to the external device, when an abnormal state of the biological information is detected, and
the first quantity of data is smaller than the second quantity of data.

8. The method of claim 7, wherein the transmitting the biologic information comprises transmitting all of the biological information to the external device using the selected second transmission mode.

9. The method of claim 7, wherein the transmitting the biologic information comprises transmitting the biological information to the external device using at least one of the first transmission mode and second transmission mode while equalizing a transmission frequency of each portion of the biological information.

10. The method of claim 1, wherein the transmitting the biological information comprises transmitting the biological information to the external device by controlling a transmission frequency of at least one portion of the biological information based on a state of the biological information.

11. The method of claim 1, wherein the selecting the transmission mode comprises:
comparing values indicating the state of the biological information to threshold values and
selecting one of the transmission modes from the plurality of transmission modes according to a result of the comparing the values indicating the state of the biological information to the threshold values.

12. The method of claim 11, wherein the threshold values are determined by the external device.

13. The method of claim 11, wherein the values indicating the state of the biological information comprise at least one selected from a group consisting of an electrocardiograph, a photoplethysmograph, a heart rate, a blood oxygen saturation, an intensity of a wireless signal transmitted to the external device and a maintaining time of the selected transmission mode.

14. The method of claim 11, wherein the threshold values comprise at least one selected from a group consisting of maximum heart rate, minimum heart rate, heart rate variation, minimum blood oxygen saturation, blood oxygen saturation variation, minimum wireless signal intensity and maximum transmission mode-maintaining time.

15. The method of claim 1, further comprising storing biological information which is not transmitted to the external device,
wherein the stored biological information is downloaded to the external device using a wired connected.

16. A computer program product comprising a computer readable computer program code for executing a method of transmitting biological information, and instructions for causing a computer to implement the method, the method comprising:
selecting a transmission mode from a plurality of transmission modes, each of the transmission modes transmitting different quantities of data to an external device based on a state of biological information of a user; and
transmitting the biological information of the user to the external device using the selected transmission mode.

17. An apparatus for transmitting biological information comprising:
a controller which selects a transmission mode from a plurality of transmission modes, each of the transmission modes transmitting different quantities of data to an external device based on a state of biological information of a user; and
a transmitter which transmits the biological information of the user to the external device using the selected transmission mode.
